(19) United States
(12) Patent Application Publication
(10) Pub. No.: US 2021/0043309 A1
(43) Pub. Date: Feb. 11, 2021

(54) SYSTEM AND METHOD FOR INVENTORY MANAGEMENT IN HOSPITAL

(71) Applicant: Vivek Tiwari, Mumbai (IN)

(72) Inventor: Vivek Tiwari, Mumbai (IN)

(21) Appl. No.: 16/985,876

(22) Filed: Aug. 5, 2020

Related U.S. Application Data
(60) Provisional application No. 62/903,962, filed on Sep. 23, 2019.

(30) Foreign Application Priority Data
Aug. 7, 2019 (IN) 201921031911

(51) Int. Cl. G16H 40/20; G06Q 10/08; G06N 20/00

(52) U.S. Cl. CPC G16H 40/20; G06N 20/00; G06Q 10/0875

(57) ABSTRACT

Present disclosure relates to a method (30) for inventory management in a medical facility. The method includes steps of receiving a user input (3) related to one or more medical procedures by an input unit (2), receiving and processing the user input (3) by a processing unit (22), and based on such processing, retrieving a mapping information (11) for the one or more medical procedures and a historical information (16) for the one or more medical procedures from a memory device (10), and processing the mapping information (11) and the historical information (16) by the processing unit (22), and generating at least one of an inventory forecast (23) related to the inventory of items required by a medical facility, or a procedure forecast (24) related to number of medical procedures to be taking place in the medical facility, or combination thereof. The mapping information (11) relates to mapping between a medical procedure and an inventory of items required to carry out the medical procedure, the historical information (16) is related to consumption of items in past for the one or more medical procedures, the historical information (16) is part of a historical database (15) stored in the memory device (10).

### FIELD OF INVENTION

The present invention relates to the field of inventory management and planning and more specifically to inventory management and planning in hospital and forecasting stock requirements.

### BACKGROUND OF INVENTION

A major part of an average hospital budget accounts for medical supplies. Effectively managing medical supplies in a hospital is critical in healthcare sector. One is required to keep the inventory as lean as possible while ensuring that the required essential medical supplies are readily available all time. One of the major challenges faced by hospitals is cancellation of planned surgeries or medical procedures due to missing medical supplies. On the other side most of the new hospitals are clueless as how much medical supplies they should stock for certain number of procedures. This is due to the fact that the new hospitals do not have any historical data for projecting the medical supply stocks that would be required for the forecasted medical procedures that the hospital is expected to conduct.

There exist techniques in prior art for inventory management and forecasting the stocks. The general approaches for inventory management are focused mainly on project stock requirement based on the movement of an item in the immediate previous period. This movement may happen due to the sale of an item to the end consumer or customer. The movement may also happen due to issue of an item from central inventory for use or stocking at a particular location. For example, items may be issued from main inventory store to Operation Theatre for a particular procedure or for stocking these items at that location for future consumption. In case of manufacturing or assembly industry, items may be issued for use in a process or in an assembly line.

Generally, stock forecast is arrived by extrapolating the movement of item by help of various existing techniques based on relationship that can be used to automatically calculate forecasts of demand. This is done using demand history data. These forecast techniques are used to calculate fresh base forecast from actual demand adjusted for seasonal and period length variations. A method is specified for each forecast technique. The technique also contains specified parameters and limits which regulate the calculation performed using the method.

One such existing technique for forecasting of stocks is based on moving averages. This forecast method calculates the base forecast for the next period as the average of historic base demand for a specified number of periods. This is denoted in the equation 1 below:

F(i+1)=(D(i)+D(i−1)+ . . . +D(i−(n−1)))/n (1)

The number of periods used determines how quickly the averaging will react to changes in actual trends and how sensitive it will be to random variations. The more periods included will make the calculation method more stable from random variations, but it will also react more slowly to changes resulting from real trends.

Another existing technique for forecasting of stocks is based on Two-period weighted average. This forecast method weighs the average demand from the latest quarter (of periods included in the forecast) with the average demand for all historic periods. The weight factor is the smoothing constant for exponential smoothing, (, and 1−(, respectively. This is denoted in the equation 2 below:

F(i+1)=((i)*+M+(1−((i))*L (2)

Yet another technique exists in prior art for forecasting of stocks is based on Exponential smoothing. This forecast method weighs latest base demand value with the smoothing constant (, while the previous base forecast value is weighted with 1−). This is denoted in the equation 3 below:

F(i+1)=((i)*D(i)+(1−((i))*F(i) (3)

The value of smoothing constant (determines how quickly the forecast will react to changes in actual trends and how sensitive it will be to random variations. The lower the value, the more stable the calculation is from random variations, but it will also react more slowly to changes resulting from real trends. Smoothing constant (must be between 0 and 1).

Yet another existing method for forecasting of stocks is based on Adaptive exponential smoothing. This forecast method is similar to basic exponential smoothing in that the latest base demand value is weighted with smoothing constant (, while the previous base forecast value is weighted with 1−(. However, in adaptive exponential smoothing, the smoothing constant is recalculated every time a new forecast is made. This is denoted in the equation 4 below:

F(i+1)=((i)*D(i)+(1−((i))*F(i) (4)

The smoothing constant is recalculated using the equation 5 below:

((i)=((min.)+((max.)*(ABS(ME(i))/MAD(i)) (5)

This forecast method uses (values that are adjusted for the current systematic forecast error. A larger mean forecast error results in a higher value. This results in quicker corrections to the forecast towards reflecting actual demand.
Wherein: ((i)=Allowable smoothing constant for smoothing in period (i)
((min.)=Minimum allowable smoothing constant
((max.)=Maximum allowable smoothing constant
D(i)=Base demand for period (i)
F(i)=Base forecast for period (i)
A(n)=Average demand for (n) periods
i=Period number
n=Number of periods included in calculating the average
L=Average demand for the latest (n) periods
M=Average demand for the latest 25% periods out of the total of (n) periods
MAD(i)=Forecast MAD for period (i)
ME(i)=Mean forecast error for period (i)
ABS( )=Absolute difference, the difference without minus sign

The existing techniques in prior art has various problems. One such problem is that the stock movement does not incorporate any specific attributes like capacity utilization e.g. occupancy in a hospital at a given point of time, seasonality and/or trends or introduction of any new services which brought in additional Stock Keeping Units (SKU's) to be bought or increase in required quantity of existing SKUs in stock etc.

Another problem with the existing techniques is that usually maximum of last one year's historic sales or issue data is taken as base data for projection. Yet another problem might be issued doesn't necessarily means consumed at the end point. It may be laying as the closing stock for example stock issued from inventory but lying at production floor or in case of hospital stock issued from central inventory but lying in inventory of operating rooms.

Yet another problem with the existing techniques is use of sales data or inventory issued of the medical supplies. The sales data or the inventory issued might be different as compared to what would have been the actual consumption of medical supplies. Consumption of items is the best data because it has sustained all factors like occupancy in the hospital or introduction of any new services, disease trend etc.

Also, the healthcare industry has experienced significant change and innovation in therapeutic and procedural care for patients over the last decade. However, the supply chain supporting this new environment is virtually unchanged over the last decades and is engineered to support a fee-for-service, hospital-based provider model which ignores proven technology and best practice developments within the supply chains of other dynamic industries. The current healthcare supply chain is well behind most industry models with respect to costs, quality and services provided, thereby creating a need to re-engineer and adopt new practices and models.

The existing techniques also fails to disclose a lean supply chain system applied to healthcare with mass customized order fulfillment, closed loop inventory management and feedback systems, real-time monitoring and data flows to connect the healthcare supply chain from patient to manufacturer, and machine learning based feedback for both clinicians and hospital administrators. The existing techniques also lacks in accurately forecasting the stock requirement in a hospital.

In one prior art patent CN109509030 titled “A Method for Sales Forecast method and its training method of model, device and electronic system”, it is disclosed to consider the relevant sales performance of sales volume of commodity, and may influence the surface of all kinds of off takes. Thus, the prediction result of Sales Volume of Commodity is more objective, accurate, helps to improve sales volume and capital turnover flexibility ratio. However, the prior art fails to disclose proper use of the sales data for training of the artificial intelligent model and forecasting of the stock requirements.

Another prior art KR101939106B1, titled “Inventory management and method using common prediction model” discloses inventory management by a server or a forecast demand using the stored predictive model to a public server, to minimize the portion of the people involved in inventory management. It shares predictive models to perform machine learning. In addition, statistical learning techniques can be used, such as time series analysis, regression analysis, to classify learning techniques may be used, such as SVM, Naive Bayesian, Decision Tree. However, the prior art does not teach forecasting based on consumption of items and therefore is not accurate.

Another prior art exists US 2019/0172012 A1, titled “Healthcare supply chain management systems, methods, and computer program products.” The prior art fails to disclose how to optimally managing the supply of stocks in the hospital This prior art does not disclose the use of consumption data of the stock inventory and focuses more on the inventory issued rather than the actual consumption.

Therefore, a system and method for inventory management in hospital for forecasting of stocks is required to overcome the problems in the above-mentioned existing techniques in prior art.

### OBJECTIVE OF INVENTION

The objective of the invention is to obtain the information regarding the overall stock requirement in a hospital. Specifically, the invention focuses on the aspect of the stock consumption for various procedures performed in the hospital. Thereby, the invention facilitates accurate measurement to forecast the stock requirement in the hospital.

### SUMMARY OF INVENTION

The objective of the invention is achieved by a method for Inventory management for a Hospital according to claim 1.

The method includes steps of receiving a user input related to one or more medical procedures by an input unit, receiving and processing the user input by a processing unit, and based on such processing, retrieving a mapping information for the one or more medical procedures and a historical information for the one or more medical procedures from a memory device, processing the mapping information and the historical information by the processing unit, and generating at least one of an inventory forecast related to the inventory of items required by a medical facility, or a procedure forecast related to number of medical procedures to be taking place in the medical facility, or combination thereof. The mapping information relates mapping between a medical procedure and an inventory of items required to carry out the medical procedure, and the historical information is related to consumption of items in past for the one or more medical procedures. The historical information is part of a historical database stored in the memory device.

According to one embodiment of the method, wherein the forecast is generated for a predefined period.

According to another embodiment of the method, wherein the historical information is for a specific medical facility for which a specific medical facility forecast is generated.

According to yet another embodiment of the method, wherein the historical information is for more than one medical facilities.

According to one embodiment of the method, wherein the historical information is for more than one medical facility located in a predefined geographical area.

According to another embodiment of the method, the method comprising steps of receiving a geographic area information related to a location of the medical facility by the processing unit, processing the geographic area information, the mapping information for the one or more medical procedures and the historical information for the one or more medical procedures, and generating the at least one of the inventory forecast related to the inventory of items required by the medical facility, the procedure forecast related to the number of procedures to be taking place in the medical facility, or a disease outbreak forecast for the geographic area, or combination thereof.

According to yet another embodiment of the method, the method includes the steps of receiving an additional input by the input unit, receiving the additional input by the processing unit, processing the additional input along with the geographical area information, the mapping information for the one or more medical procedures and the historical information for the one or more medical procedures by the processing unit, and generating the at least one of the inventory forecast, or the procedure forecast, or combination thereof. The additional input includes at least one of a medical facility infrastructure information, a medical speciality information, or a staff headcount information, or combination thereof. The medical facility infrastructure information is defined by a physical capacity related data of the medical facility to carry out various procedures, the medical speciality information is related to various specialities the medical facility is having, and the staff headcount information relates to number of staff of various categories are involved to carry out various procedures at the medical facility.

According to one embodiment of the method, the method includes steps of retrieving at least one of a seasonality of medical requirements data or a trend related data, or combination thereof, by the processing unit from the memory device, and processing the seasonality of medical requirements data or the trend related data along with the mapping information for the one or medical procedures and the historical information for the one or more medical procedures, and generating the forecast.

According to yet another embodiment of the method, wherein the historical information further comprises at least one of the number of procedures done in the past by the medical facility, a number of surgeons the medical facility engages, or an availability of operating rooms, or combination thereof.

According to one embodiment of the method, the method includes steps of receiving an editing input by the processing unit by the input unit, retrieving the mapping information by the processing unit from the memory device, processing the mapping information and the editing input by the processing unit, generating an updated mapping information, and sending the updated mapping information to the memory device.

According to another embodiment of the method, wherein the user input includes an expected number of one or more types of medical procedures to be carried out.

According to yet another embodiment of the method, wherein the user input includes an identification information related to more than one medical facilities for whom the forecast is to be generated. The method further includes steps of processing the user input by the processing unit, and based on that, retrieving and processing the mapping information for the one or more medical procedures from the one or more medical facilities and the historical information for the one or more medical procedures by the processing unit, and generating at least one of a cumulative inventory forecast related to the inventory of items required by the medical facilities, or a cumulative procedure forecast related to the number of procedures to be taking place in the medical facilities, or combination thereof, by the processing unit.

According to one embodiment of the method, the method includes the step of processing the mapping information by the processing unit, and accordingly retrieving a current stock information from a stock database stored in the memory device, wherein current stock information is related to quantities of each inventory items in the stock. The method further includes step of processing the current stock information along with the at least one of an inventory forecast, or a procedure forecast, or combination thereof, by the processing unit, and generating a list of items required to be ordered. The stock database (18) is defined by quantities of each items present in the inventory.

According to another embodiment of the method, the method includes the steps of receiving one or more threshold data for one or more thresholds of quantity of an item in the inventory, and the current stock information by the processing unit from the memory device, and processing the one or more threshold data and the current stock information by the processing unit and updating an indicator for quantities of each of the items in the stock database.

According to yet another embodiment of the method, the method includes steps of receiving the threshold data and the current stock information from the memory device, and processing the threshold data and the current stock information, and generating one or more reminders for making an order for the list of items on predefined intervals.

According to one embodiment of the method, wherein the memory device further stores a delivery data, and a lead time data, and the method includes steps of processing the mapping information for the one or more medical procedures, delivery data, and the lead time data, and the historical information for the one or more medical procedures along with the user input by the processing unit, and generating a safety inventory forecast related to the inventory of items required by the medical facility to be kept as buffer for unforeseen demands for one or more procedures. The historical information includes at least one of a consumption deviation data related to deviation in consumption of inventory items in past, or a requirement variation data related to deviation between the inventory forecast and the order placed in past, or combination thereof. The delivery data is related to delivery constraints related to probability of insufficient delivery of inventory items or quality deficiency in the delivery of inventory items or combination thereof. The lead time data is defined as an expected delay for a future delivery of inventory, or a deviation in delay time for inventory delivery in past, or combination thereof.

According to another embodiment of the method, wherein the memory device stores a relational information to relate a medical speciality to more than one procedure. The method further includes steps of retrieving the relational information for one or more specialities, the mapping information for the one or more medical procedures and the historical information for the one or more medical procedures from the memory device by the processing unit, and further processing them, and generating the at least one of the inventory forecast related to the inventory of items required by one or more specialities of the medical facility, or a procedure forecast related to the number of procedures to be taking place in one or more specialties of the medical facility, or combination thereof.

According to yet another embodiment of the method, wherein the processing unit uses a decision-tree-based ensemble Machine Learning mechanism to generate the forecasts.

According to one embodiment of the method, wherein the decision-tree-based ensemble Machine Learning mechanism is an XGBoost mechanism.

### BRIEF DESCRIPTION OF DRAWINGS

FIG. 1 illustrates the system for Inventory management in the hospital to forecast stock requirement of the hospital.

The figures depict embodiments of the disclosure for purposes of illustration only. One skilled in the art will readily recognize from the following description that alternative embodiments illustrated herein may be employed without departing from the principles of the disclosure described herein.

### DETAILED DESCRIPTION

The best and other modes for carrying out the present invention are presented in terms of the embodiments, herein depicted in drawings provided. The embodiments are described herein for illustrative purposes and are subject to many variations. It is understood that various omissions and substitutions of equivalents are contemplated as circumstances may suggest or render expedient, but are intended to cover the application or implementation without departing from the spirit or scope of the present invention. Further, it is to be understood that the phraseology and terminology employed herein are for the purpose of the description and should not be regarded as limiting. Any heading utilized within this description is for convenience only and has no legal or limiting effect.

The terms “a” and “an” herein do not denote a limitation of quantity, but rather denote the presence of at least one of the referenced items.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more sub-systems or elements or structures or components preceded by “comprises . . . a” does not, without more constraints, preclude the existence of other, sub-systems, elements, structures, components, additional sub-systems, additional elements, additional structures or additional components. Appearances of the phrase “in an embodiment”, “in another embodiment” and similar language throughout this specification may, but not necessarily do, all refer to the same embodiment.

The invention focuses on providing a system and method of inventory management in a hospital for forecasting stock requirements in the hospital. The invention focuses on achieving the results by utilizing machine learning techniques for forecasting the future stock requirements. One of the major issues that is observed with respect to inventory management is that emerging hospitals are clueless regarding the quantity i.e. how much medical supplies they should stock for certain number of procedures. Further, the cancellation of planned surgeries or medical procedures due to missing medical supplies are one of the major reasons which leads to degraded performance of the hospitals. There are several inventory management solutions those are presently available address the aforementioned issues partially but none of the solutions concentrate on the aspect of “the consumed stock” of the hospital. The present invention provides a system for inventory management in a hospital for forecasting stocks requirement.

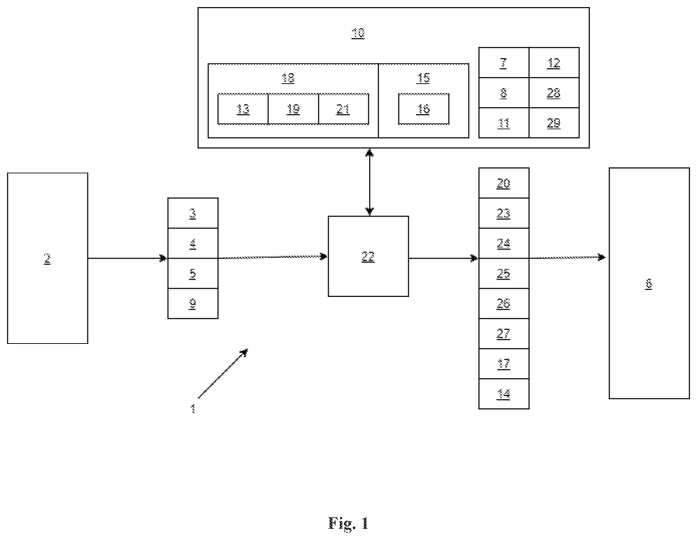

In one implementation of the invention, a mechanism is provided to manage the inventory in the hospital, and more specifically predict the stock to be consumed in the hospital for various procedures, or various medical procedures to be carried out in the hospital. One such implementation of the invention is presented in FIG. 1.

FIG. 1 shows the mechanism for predicting the stock from the system 1 for Inventory management. The system 1 includes an input unit 2, memory device 10, and processing unit 22.

The input unit 2 relates to a hardware component such as keyboard, mouse, etc which accepts the data from the user, or any other kind of sensors which can receive gestures as input, or any other sensors which automatically generates data to be used by the processing unit, such as location sensors.

The memory device is another hardware component such as Read-Only-Memory (ROM), flash memory etc, which stores and retrieves the data by the user.

The processing unit relates to another hardware component which is capable for processing any data or information received by them. In certain embodiments, these processing unit can be part of any regularly devices, such as laptops, mobile devices, servers, etc. The processing unit 22 in the present invention is dedicated to be in communicative coupling to the input unit, memory device/s, and display. It receives data from the memory device/s and input unit, and processes the data to generate forecast, and communicate the forecast to the display.

The input unit 2 of system 1 receives a user input 3. The user input 3 is related to information with respect to medical procedures. This information can be related to various types of medical procedures being carried out, or specific procedures being carried out, or identification of a medical facility where the medical procedures being carried out. The user input 3 can also be a combination of such before-said information. Some examples of medical procedures carried out are General Surgery, Cardiac Surgery, Hernia Operation, Laser removal of power, Gyneac normal delivery, Gyneac C section, Root Canal procedure and Hipec, etc.

The input unit 2 can receive any other inputs to further refine the forecasts to be provided by the processing unit 22. The inputs which can be received are as follows: a geographic area information 4, additional information 5, and trend related data 8 and editing input 9.

The geographic area information 4 relates to a location of the medical facility such as the area where the hospital/nursing home is located. The additional information 5 includes various kinds of information such as a medical facility infrastructure information, a medical speciality information, a staff headcount information. Further, the medical facility infrastructure information is defined by a physical capacity related data of the medical facility to carry out various procedures such as operating rooms, the medical speciality information is related to various specialities the medical facility is having such as automatic medical instruments, and the staff headcount information relates to number of staff of various categories are involved to carry out various procedures at the medical facility such as surgeons. The editing input 9 relates to certain data which requires certain modifications correction of values etc.

In an alternate embodiment, the user input 3 alone is sufficient to process and generate the forecast, and no other types of inputs, as mentioned above, may be required. In furtherance, one or more inputs can be received along the user input 3 depending on the requirement of the desired forecast.

The memory devices 10 of the system 1 stores various data which are retrieved by the processing unit 22 for determining the forecasts. The data stored are a mapping information 11, the relational information 12, the stock database 18, the historical database 15, seasonality of medical requirements data 7, and trend data 8.

The mapping information 11 relates to mapping between the medical procedure and an inventory of items required to carry out the medical procedure. The relational information 12 relates a medical speciality of a medical facility to more than one procedures. The stock database 18 is defined by quantities of inventory items present in the inventory of the medical facility. The stock database 18 includes a threshold data 13, a current stock information 19, and indicator 21. The current stock information 19 relates to quantities of each inventory items in the stock in the medical facility. The threshold data 13 relates to one or more thresholds of quantity of an item required to be kept in the inventory by the medical facility. The indicator 21 is a level indicator which gives a quick visual information to the user regarding the stock available for a particular item in the inventory of the medical facility. The seasonality of medical requirements data 7 relates to infections, allergies or diseases occurred during a particular period such as fever, cold, malaria, chickenpox. The trend related data 8 relates to a particular symptoms/sickness goes in a trend in one geographical area or sometimes it may be irrespective of geographical area. One good example of trend related data is COVID 19.

The memory device 10 further stores a historical database 15 which includes a historic information 16 comprising information related to items consumed in past for the one or more medical procedures. Consumption information items may be related to contains trends, seasonality, cycle, or combination thereof which is generally different for different hospitals, clinics and nursing homes. The historic information 16 may also include a number of procedures done in the past by the medical facility, and/or a number of surgeons the medical facility engages, or an availability of operating rooms.

A processing unit 22 of the system 1 retrieves data from the input unit 2 and memory devices 10 and processes the same to generate inventory forecast 23, procedure forecast 24, specific medical facility forecast 25, disease outbreak forecast 17, safety inventory forecast 20, cumulative inventory forecast 26 and cumulative procedure forecast 27. These forecasts may be generated for a specific predefined time or multiple time intervals.

The inventory forecast 23 relates to predicting the inventory of items required by a medical facility in future. The procedure forecast 24 relates to predicting the number of medical procedures to be taking place in the medical facility in future. The specific medical facility forecast 25 relates to predicting the forecasts 23, 24 for a specific medical facility. The disease outbreak forecast 17 relates to predicting the diseases which may outbreak in near future with respect to the geographical area of a particular medical facility. The safety inventory forecast 20 relates to the inventory of items required by the medical facility to be kept as buffer for unforeseen demands for one or more procedures. The cumulative inventory forecast 26 relates to) relates to the inventory of items required by more than one medical facilities. The cumulative procedure forecast 27 relates to the number of procedures to be taking place in more than one medical facilities.

In one embodiment, the processing unit 22 employs a decision-tree-based ensemble Machine Learning mechanism to generate the forecast(s). In another embodiment, the decision-tree-based ensemble Machine Learning mechanism used is an XG Boost mechanism. Alternatively, any other mechanisms can be used such as Random Forrest, SVM, LSTM, SARIMA, Gated Recurrent Units, RNN, etc.

In an alternate embodiment, the processing unit 22 generates only inventory forecast 23 or the procedure forecasts 24, or both, and no other forecasts may be required by a user, and hence the processing unit 22 shall not be configured to any other type of forecasts.

The input unit 2 receives the user input 3, and send it to the processing unit 22. The processing unit 22 processes the user input 3, and based on such processing retrieves the mapping information 11 and the historical information 16 from memory device 10, and processes them to generate the inventory forecast 23 and the procedure forecast 29. In an alternate embodiment, the processing unit 22 may only generate either of the forecasts 23, 24.

The historical information 16 is also linked with respect to a specific facility, and the processing unit generates the specific medical facility forecast 25, which can be either of the forecasts 23, 24, or both for that specific facility. In one embodiment of the system, the system is enacted in a closed computing environment of a specific medical facility, and in such scenario the historical information 16 maybe required only for that specific medical facility. The embodiment may also be implemented in a client-server architecture, where the server has the processing unit 22, and the memory device 10 storing historical database of multiple medical facilities, however while generating the forecasts 23, 24, 25 it utilizes historical information 16 of only specific medical facility.

In an alternate embodiment, the historical information 16 shall be of the medical facilities for a predefined geographical area only. The geographical area can be a specific city, or a country, or can be a hyperlocal area in a city which can be of a predefined radius, or can be a municipal division of the city, etc. This helps in fine tuning the forecasts 23, 24 for the medical facility considering the past going for the medical facilities in that geographical area.

In current embodiment, the input unit 2 further receives a geographical area information 4 and sends it to the processing unit 22. The processing unit 22 processes this geographical area information 4 along with the mapping information 11 and the historical information 16 to additionally generate the disease outbreak forecast 17 for the geographic area, and generates more refined forecasts 23, 24 with respect to the geographical area in concern. In an alternate embodiment, the processing unit 22 do not generate all the three forecasts 23, 24, 17, rather it generates any combination of the forecasts 23, 24, 17, as required by the end user. In yet another embodiment, the geographical area information 4 shall not be required, if the fine tuning of the forecasts 23, 24 is not desired or the disease outbreak forecast 17 is not required.

Generally, the processing unit 22 generates forecast 23, 24 for a bigger range of periods. However, when a specific periods forecasts is desired, the processing unit 22 generates it only for the desired predefined period. This embodiment becomes really helpful when a medical facility may want to go in a futuristic procurement agreement for a predefined period, or human resource department may want to defined the medical practitioner required to be enrolled for a predefined period, etc.

The input unit 2 further receives the additional input 5 and sends it to the processing unit 22. The processing unit 22 processes the additional input 5 along with the geographical area information 4, the mapping information 11 and the historical information 16, and generates the inventory forecast 23, or the procedure forecast 24. It is pertinent to note that use of additional input 5, and the geographical area information 4 further refines the forecasts 23, 24 considering the infrastructure details of the medical facility and the geographical area information of it. In an alternate embodiment, the processing unit 22 may only generate either of the forecasts 23, 24. In yet another embodiment, the additional input 5 and the geographical area information 4 may not be required by the processing unit 22, if a coarse level of forecasts 23, 24 are required.

The processing unit 22 also receives and processes the seasonality of medical requirements data 7 and the trend related data 8 along with the mapping information 11 and the historical information 16, and generates the inventory forecast 23, the procedure forecast 24, and the specific medical facility forecast 25. It is pertinent to note that the seasonality of medical requirements data 7 further refines the forecasts 23, 24, 25 considering the diseases/illness such as fever, flu which affects the people during particular season of the year, while the trend related data 8 refines the forecasts 23, 24, 25 considering the diseases/illness such as COVID 19 occurred in 2019, Spanish Flu occurred in 1918 which might occur over ages but the illness remains for certain period and affects huge mass of people. In an alternate embodiment, the processing unit 22 may only generate either of the forecasts 23, 24, 25, or combination thereof, and not all of the forecasts 23, 24, 25. In yet another embodiment, the seasonality of medical requirements data 7 may not be required by the processing unit 22, if forecasts 23, 24, 25 are not required with respect to seasonality constraints. In yet another embodiment, the trend related data 8 may not be required by the processing unit 22, to generate forecast 23, 24, 25 unless there is a necessity to predict the illness which occur infrequently.

The input unit 2 further receives the editing input 9 and sends it to the processing unit 22. The processing unit 22 processes the editing input 9 along with the mapping information 11 and generates the updated mapping information 11. The generated updated mapping information 11 is sent further to the memory device. This updated mapping information 11 either edits only the part of the records which is changed, or replaces the entire mapping information 11. It is pertinent to note that the option for editing the mapping information helps to replace any faulty records or change the information according to change in protocol for a particular procedure which eventually changes the inventory requirements for handling a medical procedure. In an alternate embodiment, the mapping information is hard coded, and can only be changed changing the source code on whose execution the processing unit runs, and enables the functioning of whole system.

The user input 3 includes the identification information related to more than one medical facilities for whom the forecast 23, 24 is to be generated. The processing unit 22 processes the identification information 3 along with the mapping information 11 and the historical information 16, and generates the cumulative inventory forecast 26 related to the inventory of items required by the medical facilities, and a cumulative procedure forecast 27 related to the number of procedures to be taking place in the medical facilities. The forecast 26, 27, shall be helpful to provide a combined forecast for multiple facilities run by one group or owner. This shall be especially helpful when a group order or contract is to be raised by single owner of all the facilities. In an alternate embodiment, the processing unit 22 may only generate either of the forecasts 26, 27, or combination thereof, and not all of the forecasts 26, 27. In yet another embodiment, the identification information may not be required for multiple facilities, if the cumulative forecast 26, 27, is not required.

Based on processing of the mapping information 11, the processing unit 22 also retrieves the current stock information 19 from the stock database 18 stored in the memory device 10, and processes along with the inventory forecast 23, or the procedure forecast 24, or both and generates a list of items required to be ordered. The knowing of list of items to be ordered further helps in execution process of the ordering of inventory, which was not alone possible for the forecasts 23, 24, as based on the forecasts 23, 24, substantial efforts shall be required to match with the inventory the items already in store, and then only a list could have been created for ordering. In case a medical facility has another system in place to make an order which takes inputs as forecasts 23, 24, such list generation shall not be required.

In furtherance, the processing unit 22 receives one or more threshold data 13 for one or more thresholds of quantity of an item in the inventory, and retrieves a current stock information 19 related to quantities of each of the inventory items in stock from the stock database 18, and thereafter processes them to update an indicator 21 for quantities of each of the items in the stock database 18. In an alternate embodiment, where the requirement for indicator 21 is not there, such threshold data 13 and stock database 18 shall not be required for processing.

The processing unit 22 further generates reminders 14 for making an order for the list of items on predefined intervals also, based on processing the threshold data 13 and the current stock information 19. In an alternate embodiment, where the medical facility has a separate system of ordering and setting up reminders, the processing unit 22 is not required to carry out such processing and generating reminders.

The processing unit 22 further processes the mapping information 11, a delivery data 28, a lead time data 29, and the historical information 16 and generates the safety inventory forecast 20. The historical information 16 also includes a consumption deviation data, and/or a requirement variation data, which are relevant for generating the safety inventory forecast 20. The consumption deviation data relates to deviation in consumption of inventory items in past, and the requirement variation data relates to deviation between the inventory forecast and the order placed in past. The delivery data 28 is related to delivery constraints related to probability of insufficient delivery of inventory items or quality deficiency in the delivery of inventory items or combination thereof. The lead time data 29 is defined as an expected delay for a future delivery of inventory, or a deviation in delay time for inventory delivery in past, or combination thereof. The safety forecast 20 is sometimes important to determine, if inventory available shall be enough with respect to forecast of procedures or not. In alternate embodiment, such safety forecast 20 may not be required, if there is a manager who is manually checking and determining based on his experience and forecasts 23, 24, whether the stock is safely enough for future procedures to be carried out.

When a medical facility has multiple specialities, and super specialities, like ENT, Urology, etc., each speciality may be managed separately, and may require a separate forecast. The system shown in FIG. 1 also takes care of such requirements by giving the forecast to the speciality level. The processing unit 22 retrieves and process a relational information 12 which relates a medical speciality to more than one procedures, for one or more specialities, the mapping information 11 and the historical information 16, and generates the inventory forecast 23 related to the inventory of items required by one or more specialities of the medical facility, or a procedure forecast 24 related to the number of procedures to be taking place in one or more specialities of the medical facility. In an alternate embodiment, either of the inventory forecast 23, or the procedure forecast 24 is carried out. In yet alternate embodiment, where the hospital doesn't have such department division, such forecasts may not be required.

While specific language has been used to describe the invention, any limitations arising on account of the same are not intended. As would be apparent to a person skilled in the art, various working modifications may be made to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

### LIST OF REFERENCE NUMERALS

1—System

2—Input unit

3—User input

4—Geographic area information

5—Additional input

6—Display Unit

7—Seasonality of medical requirements data

8—Trend related data

9—Editing input

10—Memory device

11—Mapping information

12—Relational information

13—Threshold data

14—Reminders

15—Historical database

16—Historical information

17—Disease outbreak forecast

18—Stock database

19—Current stock information

20—Safety inventory forecast

21—Indicator

22—Processing unit

23—Inventory forecast

24—Procedure forecast

25—Specific medical facility forecast

26—Cumulative inventory forecast

27—Cumulative procedure forecast

28—Delivery data

29—Lead time data

1. A method for Inventory management for a hospital, the method comprising:
receiving a user input (3) related to one or more medical procedures by an input unit (2);
receiving and processing the user input (3) by a processing unit (22), and based on such processing, retrieving a mapping information (11) for the one or more medical procedures and a historical information (16) for the one or more medical procedures from a memory device (10);
processing the mapping information (11) and the historical information (16) by the processing unit (22), and generating at least one of an inventory forecast (23) related to the inventory of items required by a medical facility, or a procedure forecast (24) related to number of medical procedures to be taking place in the medical facility, or combination thereof,
wherein the mapping information (11) relates mapping between a medical procedure and an inventory of items required to carry out the medical procedure, the historical information (16) is related to consumption of items in past for the one or more medical procedures, the historical information (16) is part of a historical database (15) stored in the memory device (10).

2. The method according to claim 1, wherein the forecast (23, 24) is generated for a predefined period.

3. The method according to the claim 1, wherein the historical information (16) is for a specific medical facility for which a specific medical facility forecast (25) is generated.

4. The method according to the claim 1, wherein the historical information (16) is for more than one medical facilities.

5. The method according to the claim 4, wherein the historical information (16) is for more than one medical facility located in a predefined geographical area.

6. The method according to the claim 5 comprising:
receiving a geographic area information (4) related to a location of the medical facility by the processing unit;
processing the geographic area information (4), the mapping information (11) for the one or more medical procedures and the historical information (16) for the one or more medical procedures; and
generating the at least one of the inventory forecast (23) related to the inventory of items required by the medical facility, the procedure forecast (24) related to the number of procedures to be taking place in the medical facility, or a disease outbreak forecast (17) for the geographic area, or combination thereof.

7. The method according to the claim 5 comprising:
receiving an additional input (5) by the input unit (2), wherein the additional input (5) comprising at least one of a medical facility infrastructure information, a medical speciality information, or a staff headcount information, or combination thereof;
receiving the additional input (5) by the processing unit (22);
processing the additional input (5) along with the geographical area information (4), the mapping information (11) for the one or more medical procedures and the historical information (16) for the one or more medical procedures by the processing unit (22); and
generating the at least one of the inventory forecast (23) related to the inventory of items required by the medical facility, or the procedure forecast (24) related to the number of procedures to be taking place in the medical facility, or combination thereof,
wherein the medical facility infrastructure information is defined by a physical capacity related data of the medical facility to carry out various procedures, the medical speciality information is related to various specialities the medical facility is having, and the staff headcount information relates to number of staff of various categories are involved to carry out various procedures at the medical facility.

8. The method according to the claim 1 comprising:
retrieving at least one of a seasonality of medical requirements data (7) or a trend related data (8), or combination thereof, by the processing unit (22) from the memory device (10); and
processing the seasonality of medical requirements data (7) or the trend related data (8) along with the mapping information (11) for the one or medical procedures and the historical information (16) for the one or more medical procedures, and generating the forecast (23, 24, 25).

9. The method according to the claim 1, wherein the historical information (16) further comprises at least one of the number of procedures done in the past by the medical facility, a number of surgeons the medical facility engages, or an availability of operating rooms, or combination thereof.

10. The method according to the claim 1 comprising:
receiving an editing input (9) by the processing unit (22) by the input unit (2);
retrieving the mapping information (11) by the processing unit (22) from the memory device (10);
processing the mapping information (11) and the editing input (9) by the processing unit (22), and generating an updated mapping information (11); and
sending the updated mapping information (11) to the memory device (10).

11. The method according to the claim 1, wherein the user input (3) comprises an expected number of one or more types of medical procedures to be carried out.

12. The method according to the claim 1, wherein the user input (3) includes an identification information related to more than one medical facilities for whom the forecast (23, 24) is to be generated, the method further comprising:
processing the user input (3) by the processing unit (22), and based on that retrieving and processing the mapping information (11) for the one or more medical procedures from the one or more medical facilities and the historical information (16) for the one or more medical procedures by the processing unit (22); and
generating at least one of a cumulative inventory forecast (26) related to the inventory of items required by the medical facilities, or a cumulative procedure forecast(27) related to the number of procedures to be taking place in the medical facilities, or combination thereof, by the processing unit (22).

13. The method according to the claim 1,
processing the mapping information (11) by the processing unit (22), and accordingly retrieving a current stock information (19) from a stock database (18) stored in the memory device (10), wherein current stock information (19) is related to quantities of each inventory items in the stock; and
processing the current stock information (19) along with the at least one of an inventory forecast (23), or a procedure forecast (24), or combination thereof, by the processing unit (22), and generating a list of items required to be ordered,
wherein the stock database (18) is defined by quantities of each items present in the inventory.

14. The method according to the claim 1 comprising:
receiving one or more threshold data (13) for one or more thresholds of quantity of an item in the inventory, and the current stock information (19) by the processing unit (22) from the memory device (10); and
processing the one or more threshold data (13) and the current stock information (19) by the processing unit (22) and updating an indicator (21) for quantities of each of the items in the stock database (18).

15. The method according to the claim 14 comprising:
receiving the threshold data (13) and the current stock information (19) from the memory device (10); and
processing the threshold data (13) and the current stock information (19), and generating one or more reminders (14) for making an order for the list of items on predefined intervals.

16. The method according to the claim 1, wherein the memory device (10) is further adapted to store a delivery data (28), and a lead time data (29), the method comprising:
processing the mapping information (11) for the one or more medical procedures, the delivery data (28), the lead time data (29), and the historical information (16) for the one or more medical procedures along with the user input (3) by the processing unit (22), and generating a safety inventory forecast (20) related to the inventory of items required by the medical facility to be kept as buffer for unforeseen demands for one or more procedures, wherein the historical information (16) further comprises at least one of a consumption deviation data related to deviation in consumption of inventory items in past, or a requirement variation data related to deviation between the inventory forecast and the order placed in past, or combination thereof,
wherein the delivery data (28) is related to delivery constraints related to probability of insufficient delivery of inventory items or quality deficiency in the delivery of inventory items or combination thereof, and the lead time data (29) is defined as an expected delay for a future delivery of inventory, or a deviation in delay time for inventory delivery in past, or combination thereof.

17. The method according to the claim 1, wherein the memory device (10) is further adapted to store a relational information (12) to relate a medical speciality to more than one procedure, the method further comprising:
retrieving the relational information (12) for one or more specialities, the mapping information (11) for the one or more medical procedures and the historical information (16) for the one or more medical procedures from the memory device (10) by the processing unit (22), and further processing them, and generating the at least one of the inventory forecast (23) related to the inventory of items required by one or more specialities of the medical facility, or a procedure forecast (24) related to the number of procedures to be taking place in one or more specialities of the medical facility, or combination thereof.

18. The method according to the claim 1, wherein the processing unit (22) uses a decision-tree-based ensemble Machine Learning mechanism to generate the forecasts (23, 24, 25).

19. The method according to claim 18, wherein the decision-tree-based ensemble Machine Learning mechanism is an XGBoost mechanism.

20. A computer program product stored on a non-transitory storage device, which one execution onto a processing unit (22), enable the processing unit (22), a memory device (10), and an input unit (2) to cooperate together to perform the steps for:
receiving a user input (3) related to one or more medical procedures by an input unit (2);
receiving and processing the user input (3) by a processing unit (22), and based on such processing, retrieving a mapping information (11) for the one or more medical procedures and a historical information (16) for the one or more medical procedures from a memory device (10);
processing the mapping information (11) and the historical information (16) by the processing unit (22), and generating at least one of an inventory forecast (23) related to the inventory of items required by a medical facility, or a procedure forecast (24) related to number of medical procedures to be taking place in the medical facility, or combination thereof,
Wherein the mapping information (11) relates mapping between a medical procedure and an inventory of items required to carry out the medical procedure, the historical information (16) is related to consumption of items in past for the one or more medical procedures, the historical information (16) is part of a historical database (15) stored in the memory device (10).
